(19) United States
(12) Patent Application Publication
(10) Pub. No.: US 2024/0139543 A1
(43) Pub. Date: May 2, 2024

(54) PRE-CALCULATED COLLIMATOR MODEL FOR DOSE CALCULATION SOURCE MODELING

(71) Applicant: Siemens Healthineers International AG, Steinhausen (CH)

(72) Inventors: Petri HILTUNEN, Espoo (FI); Ronan ROCHFORD, Espoo (FI); Ari HARJU, Espoo (FI); Jouko LEHTOMAKI, Espoo (FI)

(73) Assignee: Siemens Healthineers International AG, Steinhausen (CH)

(21) Appl. No.: 17/978,693

(22) Filed: Nov. 1, 2022

(51) Int. Cl. A61N 5/10

(52) U.S. Cl. CPC A61N 5/1031; A61N 5/1045; A61N 2005/1034

(57) ABSTRACT

Embodiments described herein derive improvements to a collimator model of a multi-leaf collimator (MLC) for a dose calculation source model. The MLC has movable leaves formed of an attenuating material for a beam of radiation. The collimation model includes a 3D geometric model of the MLC. The system determines a plurality of attenuation lengths at leaf of the MLC in attenuating the beam of radiation. The beam of radiation includes a plurality of beam segments, and the plurality of attenuation lengths at leaf may be based on distance that the beamlet travels in leaf along respective beam segments. The system retrieves pre-calculated beam spectrum data for the beam of radiation, and may receive data representing the input spectrum for the beam of radiation. The system applies these data to calculate adjustments to the dose calculation source model to take into account beam hardening along the MLC geometry.

### TECHNICAL FIELD

This application relates generally to modeling the radiation providing radiotherapy treatment and calculation of dose deposition for treatment planning.

### BACKGROUND

Radiation therapy is a treatment using ionizing radiation for a specific target tissue, such as a cancerous tumor. In planning treatment, a prescribing physician may identify a target location (e.g., patient's organ to be treated or tumor to be eradicated) and a corresponding dosage. Generally, determining how the dosage is delivered to the patient's tissue can be sub-divided into at least two tasks: modeling the radiation beam produced by a linear accelerator providing the radiation therapy (sometimes called source modeling), and calculating/modeling the dosage received by the patient (sometimes called dose calculation, also referred to as dosimetry). In many external photon beam radiation therapy systems, calculating the shape or fluence of a radiation beam exiting a multi-leaf collimator (“MLC”) is an important aspect of treatment planning.

### SUMMARY

There is a need for improved methods of beam tracking and dose calculation that can provide both accuracy and speed for treatment planning of radiation therapy. There is a particular need for systems and methods for planning radiation therapy that take into account changes to the radiation therapy beam other than attenuation during transmission through an MLC or other collimator.

Conventional approaches for calculating the shape or fluence of a beam transmitted through an MLC or other collimator may not take into account certain changes to the radiation beam other than attenuation during transmission through the MLC or other collimator. Further, conventional approaches may not take into account changes to the spectrum and angle distribution of the beam in transmission through MLC. Disclosed systems and methods attempt to improve methods for planning radiotherapy using a radiation system including an MLC that takes into account changes to beam spectrum such as hardening the radiation beam. In various embodiments, multi-leaf collimators are modeled for planning collimation apertures and for planning a radiation intensity profile or fluence map.

In an embodiment, a processor receives a dose calculation source model including a collimator model with three-dimensional (3D) geometry data for a multi-leaf collimator (MLC). Leaves of the MLC are comprised of an attenuating material for a beam of radiation. The 3D geometry data includes a plurality of attenuation lengths at leaf of the MLC leaves in attenuating the beam of radiation. The processor retrieves beam spectrum data for the beam of radiation corresponding to the attenuating material for the beam of radiation. The processor adjusts the dose calculation source model based on the beam spectrum data and based on the plurality of attenuation lengths at leaf of the MLC leaves in attenuating the beam of radiation. In various embodiments, the adjusted dose calculation source model takes into account hardening of the radiation beam in the MLC leaves in attenuating the beam of radiation.

In an embodiment, the radiation beam includes a plurality of beam segments or beamlets. Each of the plurality of attenuation lengths at leaf of the MLC in attenuating the beam of radiation is based on distance that the beamlet travels in leaf along the respective beam segment.

In an embodiment, the beam spectrum data is pre-calculated data. The beam spectrum data may be a look-ups data set. The look-ups data set may include attenuated spectrum data for a plurality of attenuation lengths intervals of MLC attenuating material. In an embodiment, in adjusting the dose calculation source model, depending on the attenuation lengths at leaf of MLC leaf material along the respective beam segment an attenuated spectrum can be obtained from an appropriate interval of attenuation length in the beam spectrum data set.

In disclosed embodiments, the processor receives data representing the input spectrum for the radiation beam. In an embodiment, this data includes an input spectrum for each respective beam segment of the radiation beam. The processor adjusts the dose calculation source model based on the beam spectrum data and based on the input spectrum for each respective beam segment of the radiation beam. The adjusted dose calculation source model takes into account hardening of the input spectrum of the radiation beam in interactions with the MLC leaf material in calculating changes to spectrum and angle distribution of the radiation beam.

In an embodiment, a method comprises receiving, by a processor, a dose calculation source model including three-dimensional (3D) geometry data for a multi-leaf collimator (MLC), wherein leaves of the MLC are comprised of an attenuating material for a beam of radiation; determining, by the processor, a plurality of attenuation lengths at leaf of the MLC in attenuating the beam of radiation based upon the dose calculation source model including the 3D geometry data for the MLC; retrieving, by the processor, beam spectrum data for the beam of radiation corresponding to the attenuating material for the beam of radiation; and adjusting, by the processor, the dose calculation source model based on the beam spectrum data and based on the plurality of attenuation lengths at leaf of the MLC leaves in attenuating the beam of radiation.

In an embodiment, a system comprises a server comprising a processor and a non-transitory computer-readable medium containing instructions that when executed by the processor causes the processor to perform operations comprising: receive a dose calculation source model including three-dimensional (3D) geometry data for a multi-leaf collimator (MLC), wherein leaves of the MLC are comprised of an attenuating material for a beam of radiation; determine a plurality of attenuation lengths at leaf of the MLC in attenuating the beam of radiation based upon the dose calculation source model including the 3D geometry data the MLC; retrieve, beam spectrum data for the beam of radiation corresponding to the attenuating material for the beam of radiation; and adjust the dose calculation source model based on the beam spectrum data and based on the plurality of attenuation lengths at leaf of the MLC leaves in attenuating the beam of radiation.

### BRIEF DESCRIPTION OF THE DRAWINGS

Non-limiting embodiments of the present disclosure are described by way of example with reference to the accompanying figures, which are schematic and are not intended to be drawn to scale. Unless indicated as representing the background art, the figures represent aspects of the disclosure.

FIG. 1 illustrates components of a radiation therapy system with pre-calculated dose calculation source model, according to an embodiment.

FIG. 2 illustrates a photon collimation system in a radiation treatment system, according to an embodiment.

FIG. 3 illustrates schematically modulation of a photon radiation beam via MLC collimator in a radiation treatment system, according to an embodiment.

FIG. 4 illustrates a flow diagram of a process for collimator model adjustments in dose calculation source modeling, according to an embodiment.

FIG. 5 illustrates a flow diagram of a process for collimator model adjustments in dose calculation source modeling, according to an embodiment.

FIG. 6 illustrates a block diagram of a platform for collimator model adjustments in dose calculation source modeling, according to an embodiment.

FIG. 7 illustrates schematically interaction of beam segments of a photon radiation beam with MLC collimator in a radiation treatment system, according to an embodiment.

### DETAILED DESCRIPTION

Reference will now be made to the illustrative embodiments depicted in the drawings, and specific language will be used here to describe the same. It will nevertheless be understood that no limitation of the scope of the claims or this disclosure is thereby intended. Alterations and further modifications of the inventive features illustrated herein, and additional applications of the principles of the subject matter illustrated herein, which would occur to one skilled in the relevant art and having possession of this disclosure, are to be considered within the scope of the subject matter disclosed herein. Other embodiments may be used and/or other changes may be made without departing from the spirit or scope of the present disclosure. The illustrative embodiments described in the detailed description are not meant to be limiting of the subject matter presented.

In disclosed systems and methods, multi-leaf collimators are modeled for planning MLC collimation apertures and for planning a radiation intensity profile or fluence map of a radiation beam. In disclosed embodiments, an improved collimation model including a 3D geometric model of MLCs takes into account beam-hardening effects of the MLC leaves on beam shaping. In an embodiment, the 3D geometric model determines a plurality of attenuation lengths at leaf of the MLC leaves. In order to improve computational efficiency of a dose calculation source model, disclosed embodiments pre-calculate spectrum of the radiation beam, e.g., using Monte Carlo techniques, to derive accurate models of beam hardening effects of the spectrum.

For a successful outcome of patient radiation treatment it is imperative that the dose distribution in the target volume and surrounding tissues is known precisely and accurately. Treatment planning (dosimetry) is responsible for preparing the treatment prescriptions for patients receiving radiation therapy by quantitatively analyzing the energy distribution from the ionizing radiation. In conventional practice, this may achieved through the use of dosimetry empirical functions that sometimes are measured with suitable radiation detectors. Dosimetry functions may link the dose at any arbitrary point inside the patient or phantom to a known dose at a beam calibration (or reference) point. Dose or dose rate may be determined for a specific set of reference conditions, such as Depth in phantom Z, Field Size A, and Source-Surface Distance (SSD).

To achieve a desirable dose distribution in the target, collimating devices are used in a treatment machine to size, shape and/or modulate the intensity of the beam. For example, MLCs are widely used in radiotherapy machines to support various forms of treatment, including intensity-modulated radiotherapy (IMRT), 3D conformal radiation therapy (3D-CRT), volumetric modulated arc therapy (VMAT), stereotactic radiosurgery (SRS), and others. An MLC includes a plurality of pairs of beam-blocking leaves that can be independently moved in and out of a radiation beam. In use, a number of selected beam-blocking leaves are positioned in the radiation beam forming an aperture through which the unblocked beam passes. The shape and size of the aperture in the MLC define a shaped field at the target in the isocenter plane. As a radiation beam from a source passes through an MLC or other collimating device, the characteristics of the beam, including the shape or fluence of the beam, are modified and thus need be determined to provide input for a dose calculation algorithm to compute or predict the dose deposited in the treatment volume.

MLCs are considered as an essential tool for IMRT. In IMRT, each beam is divided into many small beamlets that can include varying intensities of radiation. Various dosimetric parameters can be affected when using MLC in IMRT. Such parameters include, e.g., dose in the buildup point, physical penumbra, as well as average and end leaf leakages. For an incident beam on a phantom, the absorbed dose varies with depth. There is an initial region where absorbed dose increases with depth (buildup region) after which absorbed dose decrease with depth. At each depth of a phantom, the absorbed dose is greatest on the beam central axis and decreases toward the beam edge as a function of lateral distance from the beam axis. The physical penumbra width is defined as the lateral distance between two isodose curves at a specified depth.

Monte Carlo simulations may be used to evaluate dosimetric characteristics of collimating devices. Monte Carlo simulations statistically estimate multiple possible outcomes of particular events (e.g., particle location, energy, direction, etc.) by simulating random repeated sampling of each particle. Monte Carlo simulations may simulate electrons and photons transport in modeling dosimetric characteristics of collimating devices such as physical penumbra as well as average and end leaf leakages. In disclosed embodiments, Monte Carlo simulations simulate electrons and photons transport in modeling beam hardening effects of collimating devices.

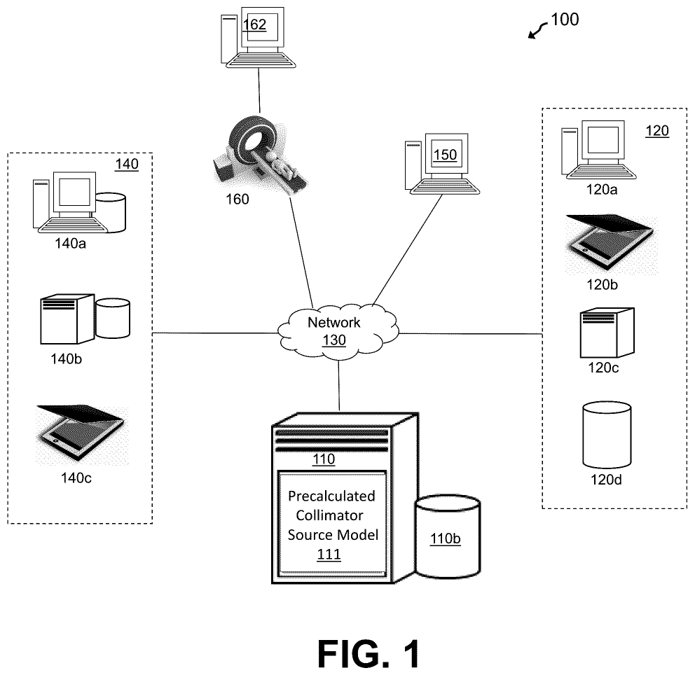

FIG. 1 illustrates components of a radiation therapy system 100 with one or more pre-calculated collimator model for dose calculation source modeling. The system 100 may include an analytics server 110a, system database 110b, pre-calculated collimator models 111, electronic data sources 120a-d (collectively electronic data sources 120), end-user devices 140a-e (collectively end-user devices 140), and an administrator computing device 150. Various features depicted in FIG. 1 may perform dose calculation source modeling. Various features depicted in FIG. 1 may belong to a radiotherapy clinic at which patients may receive radiotherapy treatment, in some cases via one or more radiotherapy machines located within the clinic (e.g., radiotherapy machine 140d).

The above-mentioned components may be connected to each other through a network 130. Examples of the network 130 may include, but are not limited to, private or public LAN, WLAN, MAN, WAN, and the Internet. The network 130 may include wired and/or wireless communications according to one or more standards and/or via one or more transport mediums. The communication over the network 130 may be performed in accordance with various communication protocols such as Transmission Control Protocol and Internet Protocol (TCP/IP), User Datagram Protocol (UDP), and IEEE communication protocols. In one example, the network 130 may include wireless communications according to Bluetooth specification sets or another standard or proprietary wireless communication protocol. In another example, the network 130 may also include communications over a cellular network, including, for example, a GSM (Global System for Mobile Communications), CDMA (Code Division Multiple Access), EDGE (Enhanced Data for Global Evolution) network.

The system 100 is not confined to the components described herein and may include additional or other components, not shown for brevity, which are to be considered within the scope of the embodiments described herein.

The analytics server 110a may generate and display an electronic platform configured to use pre-calculated collimator models and dose calculation source models 111 to identify and display dose calculation source modeling data. The electronic platform may include graphical user interfaces (GUIs) displayed on each electronic data source 120, the end-user devices 140, and/or the administrator computing device 150. An example of the electronic platform generated and hosted by the analytics server 110a may be a web-based application or a website configured to be displayed on different electronic devices, such as mobile devices, tablets, personal computer, and the like. The analytics server 110a may utilize the methods and systems described herein to optimize treatment plans and display the results on the end-user devices or adjust the configuration of one of end-user devices 140 (e.g., the radiotherapy machine 140d). The analytics server 110a may display the pre-calculated collimator models and predicted dose calculation source models on the clinic computer 120a or physician device 120b.

In a non-limiting example, a dosimetrist operating the clinic computer 120a may access the platform and further instruct the analytics server 110a to generate collimator models and dose calculation source models. In a non-limiting example, a physician operating the physician device 120b may access the platform, input patient attributes or characteristics and other data, and further instruct the analytics server 110a to optimize the patient's treatment plan (e.g., dose distribution among the patient's organs).

As described herein, radiation parameters may be or include any attributes related to using a radiotherapy machine, e.g., for treating patients at a radiotherapy clinic. Radiation parameters may include, but are not limited to, different treatment modalities, field geometry settings for external beam radiotherapy, side effect predictions, organ and/or tumor segmentation, machine therapy attributes, dosage administration attributes (e.g., dosage amount), treatment frequency, treatment timing, etc.

The analytics server 110a may host a website accessible to users operating any of the electronic devices described herein (e.g., end users), where the content presented via the various webpages may be controlled based upon each particular user's role or viewing permissions. The analytics server 110a may be any computing device comprising a processor and non-transitory machine-readable storage capable of executing the various tasks and processes described herein. Non-limiting examples of such computing devices may include workstation computers, laptop computers, server computers, and the like. While the system 100 includes a single analytics server 110a, the analytics server 110a may include any number of computing devices operating in a distributed computing environment, such as a cloud environment.

The analytics server 110a may execute software applications configured to display the electronic platform (e.g., host a website), which may generate and serve various webpages to each electronic data source 120 and/or end-user devices 140. Different users may use the website to view and/or interact with the predicted results.

The analytics server 110a may be configured to require user authentication based upon a set of user authorization credentials (e.g., username, password, biometrics, cryptographic certificate, and the like). The analytics server 110a may access the system database 110b configured to store user credentials, which the analytics server 110a may be configured to reference in order to determine whether a set of entered credentials (purportedly authenticating the user) match an appropriate set of credentials that identify and authenticate the user.

The analytics server 110a also may store data associated with each user operating one or more electronic data sources 120 and/or end-user devices 140. For instance, the analytics server 110a may indicate that a user is a medical professional whose inputs may be monitored and used as described herein.

The analytics server 110a may generate a user interface (e.g., host or present a webpage) that presents information based upon a particular user's role within the system 100. In such implementations, the user's role may be defined by data fields and input fields in user records stored in the system database 110b. The analytics server 110a may authenticate the user and may identify the user's role by executing an access directory protocol (e.g. LDAP). The analytics server 110a may generate webpage content that is customized according to the user's role defined by the user record in the system database 110b.

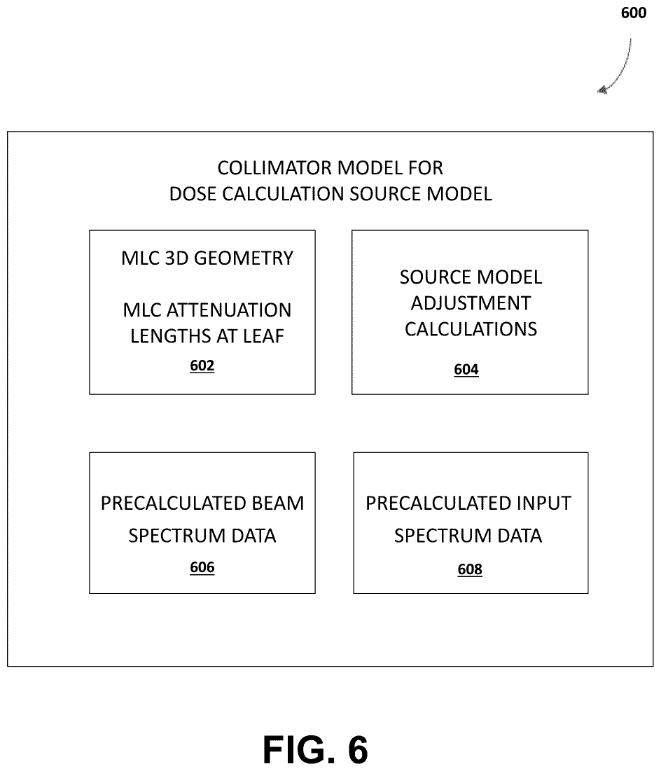

The electronic data sources 120 may represent various electronic data sources that contain, retrieve, and/or input data associated with MLC leaf geometry, attenuation lengths at leaf of MLC beam-blocking material, pre-calculated beam spectrum data, and pre-calculated input spectrum data (FIG. 6). Additionally, electronic data sources may contain, retrieve, and/or input data associated with patients and their treatment (e.g., patient data, treatment plans, and radiation parameters). For instance, the analytics server 110a may use the clinic computer 120a, physician device 120b, server 120c (associated with a physician and/or clinic), and database 120d (associated with the physician and/or the clinic) to retrieve/receive data associated with a particular patient's treatment plan.

End-user devices 140 may be any computing device comprising a processor and a non-transitory machine-readable storage medium capable of performing the various tasks and processes described herein. Non-limiting examples of an end-user device 140 may be a workstation computer, laptop computer, tablet computer, and server computer. In operation, various users may use end-user devices 140 to access the GUI operationally managed by the analytics server 110a. Specifically, the end-user devices 140 may include clinic computer 140a, clinic database 140b, clinic server 140c, a medical device, such as a CT scan machine, radiotherapy machine (e.g., a linear accelerator, cobalt machine or the like) 140d, and a clinic device 140e.

The administrator computing device 150 may represent a computing device operated by a system administrator. The administrator computing device 150 may be configured to display data retrieved and/or radiation parameters generated by the analytics server 110a (e.g., various analytic metrics and/or field geometry) where the system administrator can monitor various models 111 utilized by the analytics server 110a, electronic data sources 120, and/or end-user devices 140; review feedback; and/or facilitate training or calibration of collimator models that are maintained by the analytic server 110a.

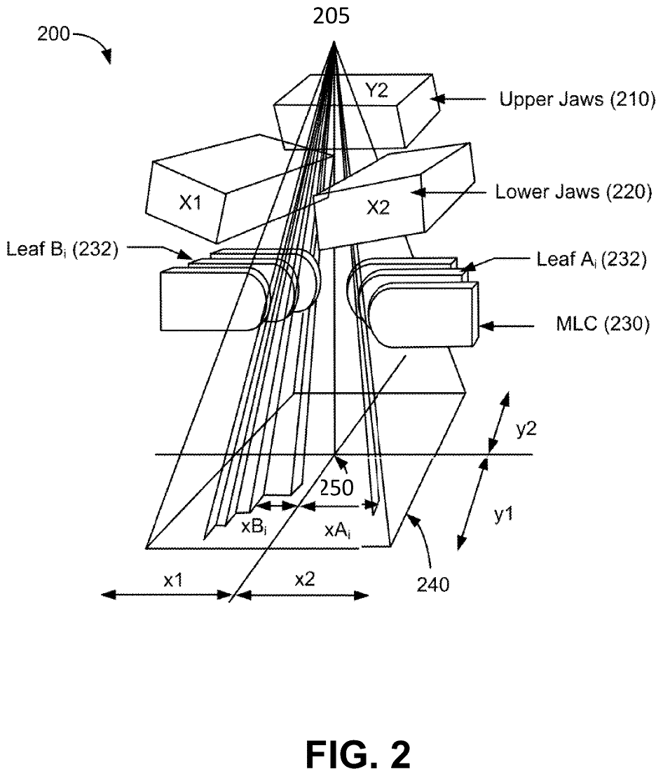

The analytics server 110a may be in communication (real-time or near real-time) with the medical device 140d, such that a server/computer hosting the medical device 140d can adjust the medical device 140d based on the radiation parameters generated by the analytics server 110a. For instance, the radiotherapy machine may adjust the collimator system (FIG. 2) based on adjusted collimator settings determined by the analytics server 110a. The analytics server 110a may transmit instructions to the radiotherapy machines indicating any number or type of radiation parameters (e.g., field geometry settings) to facilitate such adjustments.

The database 110b may store data for individual radiotherapy clinics or otherwise different sets of radiotherapy machines (e.g., located at individual radiotherapy clinics, are located in different geographical regions, treat specific types of diseases (e.g., different type of cancers), treat specific genders, etc.). An operator at a radiotherapy clinic may access an end-user device 140 located at the clinic or access an account associated with the clinic. The operator may provide an input at a user interface that causes the end-user device 140 to transmit a request to access a particular 3D dose calculation source model 111 that is associated with the clinic and/or the radiotherapy machines located within the clinic. The request may include an identifier associated with the dose calculation source model 111, the clinic, and/or the set of radiotherapy machines that the analytics server 110a may use as a key in a look-up table to identify the desired dose calculation source model 111 and to identify pre-calculated beam spectrum data and pre-calculated angle distribution data pertinent to the source model 111. The analytics server 110a may receive the request and, in some cases, after authenticating the user, identify the source model 111 via the identifier.

FIG. 2 shows schematically a photon collimation system 200 with upper jaws 210 (i.e., the Y1 and Y2 jaws; the Y1 jaw is omitted for clarity), lower jaws 220 (i.e., the X1 and X2 jaws), and an MLC 230. The field dimensions in an MLC plane 240 at an isocenter 250 are indicated. The isocenter 250 is at the intersection between the MLC plane 240 and the central axis of a beam emitted from a treatment head (not shown) of the MLC 230. The upper jaws 210, the lower jaws 220, and the leaves 232 of the MLC 230 comprise a beam-blocking material, e.g., x-ray blocking material. These structures are positioned in the treatment head to define the width of the radiation beam at a patient plane (not shown). The patient plane extends across a body of the patient (e.g., a mid-coronal plane) and is usually positioned about one meter from the x-ray source or the metal target. The axis of a gantry (not shown) is located on the patient plane, such that the distance between the metal target and the isocenter 250 remains constant when the gantry is rotated. Typically, the jaws 210 and 220 are moveable and, when fully open, define a maximum beam of about 40 cm×40 cm at the patient plane. The MLC 230 is positioned at the exit of the treatment head, to further shape the radiation beam. In various embodiments, MLC 230 use individually controllable leaves, typically thin slices of a high atomic numbered material such as tungsten, which can be moved into or out of the radiation beam under the control of system software. In an embodiment, each individual leaf can be moved independently by a motor drive.

In IMRT, beam intensity is varied across each treatment region (target) in a patient. Commonly employed techniques for intensity modulation include beam shaping (collimation) and angle of incidence (sometimes referred to as beam geometry). IMRT treatment planning typically is performed in three phases. A first phase determines a set of beam angles through which radiation is delivered. A second phase that determines an optimal radiation intensity profile, or fluence map. To deliver these intensity profiles to the patient, a third phase translates these intensity profiles to a collection of collimation apertures and corresponding intensities.

During the second phase of IMRT treatment, the intensity profile, or fluence map, is selected to ensure that certain targets receive a required amount of dose while functional organs are spared. Radiant exposure or fluence is the radiant energy received by a surface per unit area, or equivalently the irradiance of a surface, integrated over time of irradiation. Spectral exposure is the radiant exposure per unit frequency or wavelength.

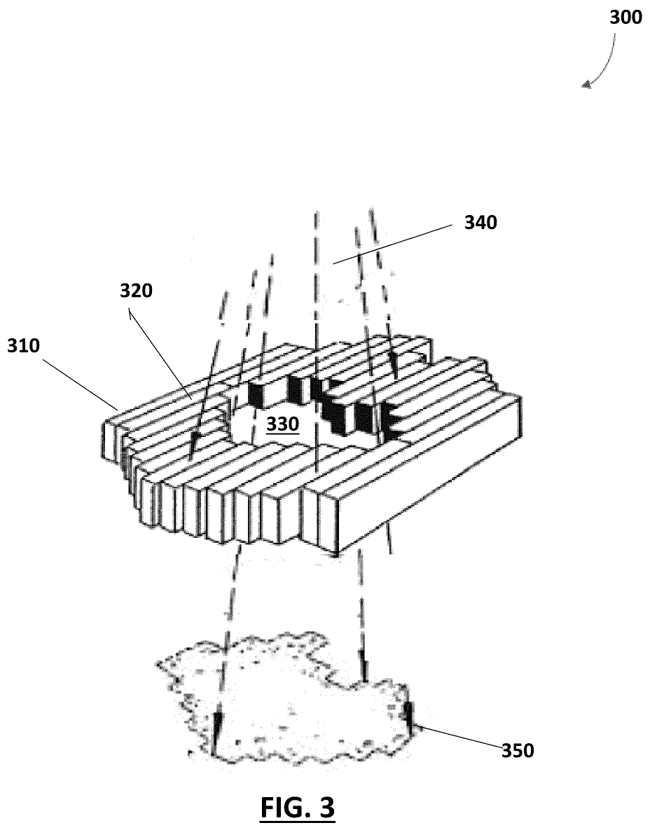

As seen in the schematic diagram of FIG. 3, MLC collimation system 300 incorporates MLCs 310 including leaves 320 that define an aperture 330. MLCs 310 modulate transmission of photon therapy beam 340, e.g., creating an irregularly shaped beam 350. In an embodiment, MLC leaves 320 comprise beam-blocking leaves that can be independently moved in and out of path of photon therapy beam 340. MLC leaves 320 are formed of an attenuating material for the photon therapy beam 340, also referred to herein as beam-blocking material. In an embodiment, MLC leaves 320 are formed of a high atomic numbered material such as tungsten or a tungsten alloy. In planning beam geometry and beam shaping of photon therapy beam 340, MLC collimation system 300 can incorporate source calculations including very complex non-uniform dose distributions.

Conventional source calculations of MLC collimator systems project the collimator geometry along the beamline onto the isocenter plane, e.g., by flattening the 3D geometry into a two-dimensional opening. The method has been improved to take into account the variable attenuation along the 3D collimator geometry. In disclosed embodiments, MLCs 310 may be modeled for planning collimation apertures and for planning a radiation intensity profile or fluence map. In disclosed embodiments, a collimation model including a 3D geometric model of MLCs 310 includes beam-hardening effects of the MLC leaves 320 on MLC beam shaping.

In an embodiment, the system 300 stores a dose calculation source model including a collimator model with three-dimensional (3D) geometry data for MLC 310. The system 310 may determine a plurality of attenuation lengths at leaf of the MLC 310 in attenuating the beam of radiation based upon the dose calculation source model including the 3D geometry data for the MLC. The system 300 may retrieve beam spectrum data for the photon therapy beam 340 corresponding to attenuating material or beam-blocking material of the MLC leaves 320. The system 300 may adjust the dose calculation source model based on the beam spectrum data and based on the attenuation lengths at leaf of the MLC leaves in attenuating the beam of radiation. In various embodiments, the adjusted dose calculation source model takes into account hardening of the radiation beam in the MLC leaves in attenuating the beam of radiation.

In an embodiment, the photon therapy beam 340 includes a plurality of beam segments or beamlets. In an embodiment, each of the plurality of attenuation lengths at leaf of the MLC 310 in attenuating the beam of radiation is based on distance that the beamlet travels in leaf along the respective beam segment.

In an embodiment, the leaves of the MLC 310 may have substantially uniform heights or physical thicknesses. In another embodiment, MLC 310 may include two or more heights or physical thicknesses. Different MLC leaves 320 may have different heights or physical thicknesses, or individual MLC leaves 320 may have multiple heights or physical thickness. In this embodiment, variations in height of the leaves 320 may be an additional factor in determining attenuation lengths at leaf of the MLC 310 in attenuating the beam of radiation.

Existing source modeling techniques make a simplifying assumption that calculates a radius adjustment of spectrum based on distance from the central axis. This adjustment is based on the input spectrum, which does not take into account effect of collimating devices on the spectrum. The system and method of the disclosure enable calculation of adjustments to beam spectrum at different locations of the field based on how the MLC structure interacts with the radiation beam. An MLC aperture may be asymmetrical in its effect on radiation beam spectrum, e.g., spectra of beam segments or beamlets.

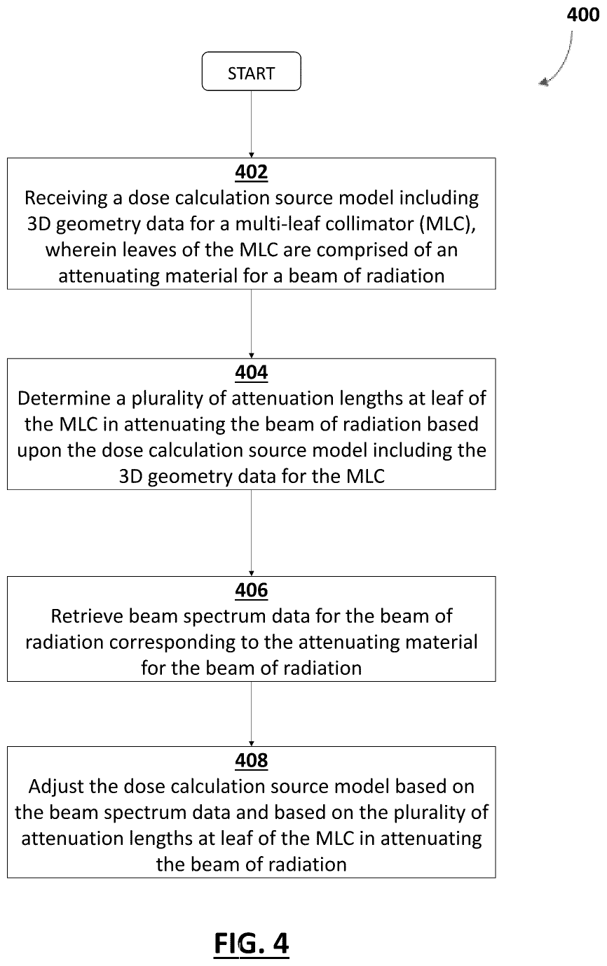

FIG. 4 illustrates a flow diagram of a process for collimator model adjustments in dose calculation source modeling. The method 400 may include steps 402-408. However, other embodiments may include additional or alternative steps, or may omit one or more steps altogether.

In an embodiment, the method 400 is executed by an analytics server, such as the analytics server described in FIG. 1. The analytics server may employ one or more processing units, including but not limited to CPUs, GPUs, or TPUs, to perform one or more steps of method 400. The CPUs, GPUs, and/or TPUs may be employed in part by the analytics server and in part by one or more other servers and/or computing devices. The servers and/or computing devices employing the processing units may be local, remote, or some combination. For example, one or more virtual machines in a cloud may employ one or more processing units, or a hybrid processing unit implementation, to perform one or more steps of method 400. However, one or more steps of method 400 may be executed by any number of computing devices or processors. For instance, one or more processors may locally perform part or all of the steps described in FIG. 4.

In step 402, the processor receives a dose calculation source model including three-dimensional (3D) geometry data for a multi-leaf collimator (MLC), in which leaves of the MLC are comprised of an attenuating material for a beam of radiation. In an embodiment, the beam of radiation comprises ionizing radiation for a radiation treatment system. In an embodiment, the MLC has movable leaves of the attenuating material to block some fraction of the beam of radiation.

In an embodiment of step 402, the attenuating material for the beam of radiation comprises a high atomic numbered material. The high atomic numbered material may comprise a tungsten alloy. Heavy metal tungsten alloys may be 90% to 97% pure tungsten in a matrix of nickel and copper or matrix of nickel and iron (e.g., tungsten alloy grades W90, W93, W95, and W97). The addition of these alloying elements improves both the ductility and machinability of these alloys over non-alloyed tungsten. MLC leaves density can be an important factor in dosimetric parameters. In various examples, MLC leaves are made of high-density tungsten alloy, e.g., with densities such as 17.5 gr/cm³, 18.0 gr/cm³, or 18.5 gr/cm³.

In step 404, the processor determines a plurality of attenuation lengths at leaf of the MIX: in attenuating the beam of radiation based upon the dose calculation source model including the 3D geometry data for the MLC.

In an embodiment of step 404, the radiation beam includes a plurality of beam segments. Each of the plurality of attenuation lengths at leaf of the MLC in attenuating the beam of radiation is based on distance that the beamlet travels in leaf along the respective beam segment.

In step 406, the processor retrieves beam spectrum data for the beam of radiation corresponding to the attenuating material for the beam of radiation. In an embodiment, the beam spectrum data comprises pre-calculated photon energy spectrum data. The pre-calculated photon energy spectrum data may include a look-ups data set for a plurality of attenuation lengths intervals of the attenuating material. In various embodiments, the pre-calculated photon energy spectrum data exhibits beam hardening characteristics corresponding to the attenuating material for the beam of radiation. In an embodiment, the pre-calculated photon energy spectrum data comprise Monte Carlo simulated spectra.

In step 408, the processor adjusts the dose calculation source model based on the beam spectrum data and based on the plurality of attenuation lengths at leaf of the MLC leaves in attenuating the beam of radiation. In an embodiment, the processor further adjusts the dose calculation source model to take into account hardening of an input spectrum of the beam of radiation in interactions with the attenuating material. In various embodiments, the processor pre-calculates change in spectrum of the beam of radiation, or determines a new spectrum of the beam of radiation, before dose calculation.

In various embodiments of method 400, the processor performs the additional step of receiving data representing the input spectrum for the beam of radiation. In an embodiment, the beam of radiation includes a plurality of beam segments, and the input spectrum data includes input spectrum and beam angle distribution data for each respective beam segment of the beam of radiation. In an embodiment, step 408 further comprises adjusting the dose calculation source model based on the plurality of attenuation lengths at leaf of the MLC and based on the input spectrum and beam angle distribution data for each respective beam segment of the beam of radiation.

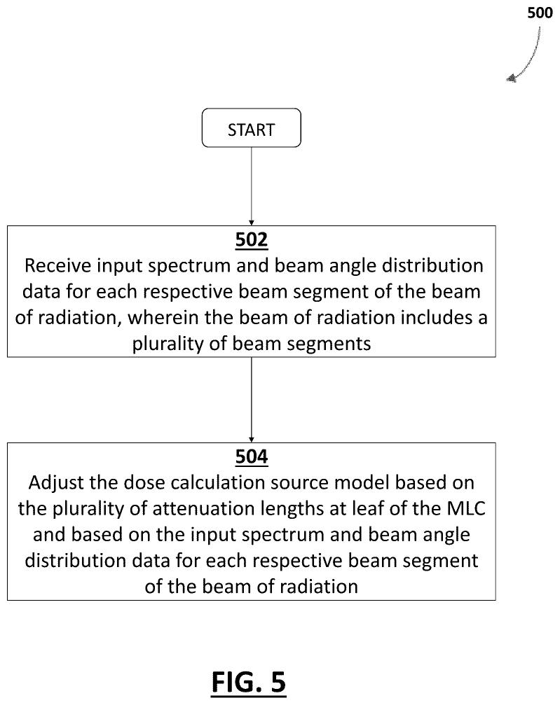

FIG. 5 illustrates a flow diagram of a process for collimator model adjustments in dose calculation source modeling. The method 500 may include steps 502-504. However, other embodiments may include additional or alternative steps, or may omit one or more steps altogether.

In an embodiment, the method 500 is executed by an analytics server, such as the analytics server described in FIG. 1. The analytics server may employ one or more processing units, including but not limited to CPUs, GPUs, or TPUs, to perform one or more steps of method 500. The CPUs, GPUs, and/or TPUs may be employed in part by the analytics server and in part by one or more other servers and/or computing devices. The servers and/or computing devices employing the processing units may be local, remote, or some combination. For example, one or more virtual machines in a cloud may employ one or more processing units, or a hybrid processing unit implementation, to perform one or more steps of method 500. However, one or more steps of method 500 may be executed by any number of computing devices or processors. For instance, one or more processors may locally perform part or all of the steps described in FIG. 5.

In step 502, the processor receives data representing the input spectrum for each respective beam segment of the beam of radiation, and beam angle distribution data for each respective beam segment, wherein the beam of radiation includes a plurality of beam segments.

In step 504, the processor adjusts the dose calculation source model based on the plurality of attenuation lengths at leaf of the MLC leaves and based on the input spectrum and angle distribution data for each respective beam segment of the beam of radiation.

FIG. 6 shows a block diagram of a collimator model for a dose calculation source model 600 according to an embodiment of the present invention. The collimator model 600 includes dose calculation source model including 3D geometry data 602, a source model adjustment calculations component 604, a pre-calculated beam spectrum data component 606, and a pre-calculated input spectrum data component 608. The dose calculation source model with 3D geometry data 602 determines a plurality of attenuation lengths at leaf of the MLC. In some embodiments, the dose calculation source model with 3D geometry data 602, the pre-calculated beam spectrum data 606, and the pre-calculated angle distribution data 608 may be stored together in a separate memory in the radiation treatment system or external to the radiation treatment system.

In some embodiments, the pre-calculated beam spectrum data 606 store look-ups data representing an accurate beam hardening model. In an embodiment, look-ups data 606 could map radiation beam spectrum at different attenuation lengths at leaf of the MLC material, so that the look-ups data includes various attenuation lengths intervals of MLC material. In an embodiment, the radiation beam includes a plurality of beam segments, and the source model adjustment calculations component 604 may be configured to calculate an adjusted dose calculation source model based on MLC attenuation lengths at leaf 602 for each beam segment of the radiation beam, and based on the pre-calculated beam spectrum data 606. Attenuation length calculation may be effected outside of a dose calculation program, and data may be stored in executable file(s) to be run by a dose calculation source model (not shown).

In an embodiment, the pre-calculated beam spectrum data 606 may display variations in photon energy spectra for open spectrum and blocked spectrum radiation beams. Blocked spectrum data may include data for various attenuation lengths intervals of MLC beam-blocking material. Blocked spectrum data may pre-calculate the effect of spectrum hardening in beam transmission through the MLC leaf material. Monte Carlo simulation may provide increased accuracy for the MLC blocked spectra. Monte Carlo simulation can take into account characteristics of the beam-blocking material.

Beam hardening effects can exhibit significant differences between low energy contributions and the high energy contributions depending on attenuation length of MLC leaf material in blocked spectrum data. Given these highly non-linear beam spectra, the use of pre-calculated beam spectrum data in dose calculation source modeling can improve efficiency and accuracy in modeling beam shaping and intensity profiles.

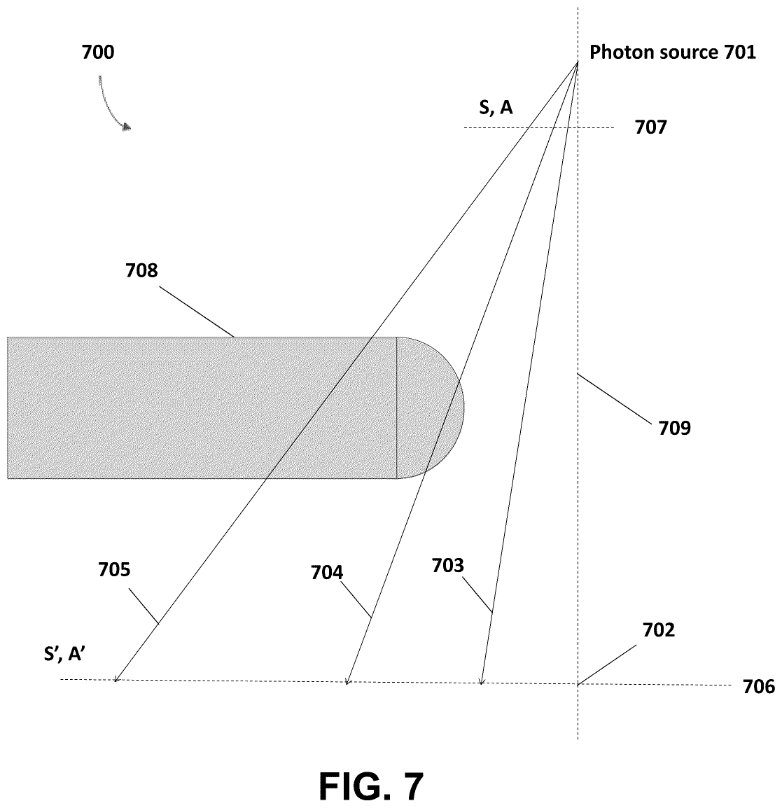

In an embodiment, the source model adjustment calculations component 604 is also configured to calculate an adjusted collimator model of the dose calculation source model based on the pre-calculated input spectrum data component 608. In an embodiment, the pre-calculated input spectrum data includes an input spectrum for each respective beam segment of the radiation beam, and includes angle distribution for each respective beam segment. The source model adjustment calculations component 604 may adjust the dose calculation source model based on the MLC attenuation lengths at leaf 602 for selected beam segments of the radiation beam. The adjusted dose calculation source model may modify the input spectrum data and angle distribution data 608 in an adjusted dose calculation source model to take into account interactions with the MLC leaf material (FIG. 7).

FIG. 7 illustrates schematically interaction of beam segments of a photon radiation beam with MLC collimator in a radiation treatment system 700. Treatment system supplies a beam of radiation from photon source 701. In an embodiment, at photon source 701, electrons from a linear accelerator hits a target. The isocenter 702 is at the intersection between the plane 706 after the MLC 708 and the central axis 709 of the beam emitted from photon source 701. Continuous radiation from the source may be divided into numerous beam segments or beamlets. FIG. 7 presents three representative beamlets of the beam. 703 is a beamlet that does not intersect with MLC leaf 708. 704 is a beamlet that travels short distance (attenuation length at leaf value) in MLC material 708. 704 is a beamlet which travels a longer distance (larger attenuation length at leaf) in MLC material 708. Each beamlet has respective spectrum S and angle distribution A at a plane 707 before the MLC 708. In plane 706 after the MLC, each beamlet has respective spectrum S′ and angle distribution A′. The spectrum S′ and angle distribution A′ for beamlet 703 at plane 706 are not changed, as there is no interaction with MLC leaf 708. Beamlets 704 and 705 have changed spectrum S′ and angle distribution A′, since the radiation has travelled through an attenuation length in MLC material. The methods of beam tracking and dose calculation of the disclosure take into account beam hardening effects for improved calculation of these changes to spectrum S′ and angle distribution A′.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of this disclosure or the claims.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The steps of a method or algorithm disclosed herein may be embodied in a processor-executable software module, which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the embodiments described herein and variations thereof. Various modifications to these embodiments will be readily apparent to those skilled in the art, and the principles defined herein may be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What is claimed is:

1. A method comprising:
receiving, by a processor, a dose calculation source model including three-dimensional (3D) geometry data for a multi-leaf collimator (MLC), wherein leaves of the MLC are comprised of an attenuating material for a beam of radiation;
determining, by the processor, a plurality of attenuation lengths at leaf of the MLC in attenuating the beam of radiation based upon the dose calculation source model including the 3D geometry data for the MLC;
retrieving, by the processor, beam spectrum data for the beam of radiation corresponding to the attenuating material for the beam of radiation; and
adjusting, by the processor, the dose calculation source model based on the beam spectrum data and based on the plurality of attenuation lengths at leaf of the MLC leaves in attenuating the beam of radiation.

2. The method of claim 1, wherein the beam of radiation includes a plurality of beam segments, wherein each of the plurality of attenuation lengths at leaf of the MLC in attenuating the beam of radiation is based on distance that the beamlet travels in leaf along the respective beam segment.

3. The method of claim 2, wherein each of the plurality of attenuation lengths at leaf of the MLC in attenuating the beam of radiation is based on distance that the beamlet travels in leaf along the respective beam segment.

4. The method of claim 1, wherein the beam spectrum data comprises pre-calculated photon energy spectrum data corresponding to the attenuating material for the beam of radiation.

5. The method of claim 4, wherein the pre-calculated photon energy spectrum data comprise a look-ups data set for a plurality of attenuation length intervals of the attenuating material.

6. The method of claim 4, wherein the pre-calculated photon energy spectrum data exhibit beam hardening characteristics corresponding to the attenuating material for the beam of radiation.

7. The method of claim 4, Wherein the pre-calculated photon energy spectrum data comprise Monte Carlo simulated spectra.

8. The method of claim 1, further comprising the step of receiving, by the processor, data representing the input spectrum and beam angle distribution of the beam of radiation.

9. The method of claim 8, wherein the beam of radiation includes a plurality of beam segments, wherein the data representing the input spectrum and beam angle distribution of the beam of radiation includes data representing the input spectrum and beam angle distribution for each respective beam segment of the beam of radiation.

10. The method of claim 9, wherein the adjusting step further comprises adjusting the dose calculation source model based on the plurality of attenuation lengths at leaf of the MLC and based on the data representing the input spectrum and beam angle distribution for each respective beam segment of the beam of radiation.

11. The method of claim 10, wherein adjusting the dose calculation source model takes into account hardening of the input spectrum of the beam of radiation in interactions with the attenuating material.

12. The method of claim 1, wherein the attenuating material for the beam of radiation comprises a high atomic numbered material.

13. The method of claim 12, wherein the high atomic numbered material comprises a tungsten alloy.

14. A system comprising:
a server comprising a processor and a non-transitory computer-readable medium containing instructions that when executed by the processor causes the processor to perform operations comprising:
receive a dose calculation source model including three-dimensional (3D) geometry data for a multi-leaf collimator (MIX), wherein leaves of the NNE are comprised of an attenuating material for a beam of radiation;
determine a plurality of attenuation lengths at leaf of the MLC in attenuating the beam of radiation based upon the dose calculation source model including the 3D geometry data the MLC;
retrieve, beam spectrum data for the beam of radiation corresponding to the attenuating material for the beam of radiation; and
adjust the dose calculation source model based on the beam spectrum data and based on the plurality of attenuation lengths at leaf of the MLC leaves in attenuating the beam of radiation.

15. The system of claim 14, wherein the beam of radiation includes a plurality of beam segments, wherein each of the plurality of attenuation lengths at leaf of the MLC in attenuating the beam of radiation is based on distance that the beamlet travels in leaf along the respective beam segment.

16. The system of claim 15, wherein the beam spectrum data comprises pre-calculated photon energy spectrum data, wherein the pre-calculated photon energy spectrum data exhibit beam hardening characteristics corresponding to the attenuating material for the beam of radiation.

17. The system of claim 16, wherein the pre-calculated photon energy spectrum data comprise a look-ups data set for a plurality of attenuation length intervals of the attenuating material.

18. The system of claim 14, wherein the instructions executed by the processor further cause the processor to receive data representing the input spectrum and beam angle distribution of the beam of radiation.

19. The system of claim 18, wherein the beam of radiation includes a plurality of beam segments, wherein the data representing the input spectrum and beam angle distribution of the beam of radiation includes data representing the input spectrum and beam angle distribution for each respective beam segment of the beam of radiation.

20. The system of claim 14, wherein adjusting the dose calculation source model takes into account hardening of the input spectrum of the beam of radiation in interactions with the attenuating material.
